(19) United States
(12) Patent Application Publication
(10) Pub. No.: US 2021/0217509 A1
(43) Pub. Date: Jul. 15, 2021

(54) Rehabilitation Support System and Method

(71) Applicant: Nippon Telegraph and Telephone Corporation, Tokyo (JP)

(72) Inventors: Rieko Sato, Tokyo (JP); Takayuki Ogasawara, Tokyo (JP); Masahiko Mukaino, Aichi (JP); Eiichi Saitoh, Aichi (JP)

(21) Appl. No.: 17/255,617

(22) PCT Filed: Jun. 17, 2019
(86) PCT No.: PCT/JP2019/023853

§ 371 Date: Dec. 23, 2020

(30) Foreign Application Priority Data
Jun. 25, 2018 (JP) 2018-119729

(51) Int. Cl. G16H 20/30; G01C 22/02; A63B 24/00

(52) U.S. Cl. CPC G16H 20/30; G01C 22/006; A63B 24/0062; G01C 22/02

(57) ABSTRACT

A first calculation unit obtains an activity amount concerning the body movement of a measurement subject from a change in physical information measured by a physical measurement unit. A second calculation unit obtains a physiological load imposed on the measurement subject from a change in physiological information measured by a physiological measurement unit. A graph generation unit generates a graph concerning the activity amount obtained by the first calculation unit and the physiological load obtained by the second calculation unit. For example, the graph generation unit sets, as a first parameter, a change in the activity amount obtained by the first calculation unit, sets, as a second parameter, a change in the physiological load obtained by the second calculation unit, and generates two-dimensional graph data with the first parameter and the second parameter.

### CROSS-REFERENCE TO RELATED APPLICATIONS

This application is a national phase entry of PCT Application No. PCT/JP2019/023853, filed on Jun. 17, 2019, which claims priority to Japanese Application No. 2018-119729, filed on Jun. 25, 2018, which applications are hereby incorporated herein by reference.

### TECHNICAL FIELD

The present invention relates to a rehabilitation support system and method for presenting the status of function restoration, problems in function restoration, the goal of function restoration, and the like.

### BACKGROUND

Patent literature 1 (Japanese Patent Laid-Open No. 2005-352686) proposes a rehabilitation management apparatus that manages the whole histories of the motor functions of a plurality of patients by analyzing information of exercises (rehabilitation exercises) done by the patents for function restoration. Furthermore, patent literature 2 (Japanese Patent Laid-Open No. 2010-108430) proposes a rehabilitation support apparatus that makes it easy to grasp, back to a desired point of time, whether the evaluation values of a number of evaluation items of rehabilitation indicate a good direction, no change, or a bad direction as a whole.

### RELATED ART LITERATURE

### Patent Literature

Patent Literature 1: Japanese Patent Laid-Open No. 2005-352686
Patent Literature 2: Japanese Patent Laid-Open No. 2010-108430

### Non-Patent Literature

Non-Patent Literature 1: Nikkei BigData, Data Market, “Stepwise Method that Provides Easy Explanation of Variable Selection Process”, [Searched on May 23, 2019], (https://business.nikkeibp.co.jp/atclbdt/15/recipe/120400035/?ST=print).

### SUMMARY

### Problem to be Solved by Embodiments of the Invention

However, the above-described conventional techniques are limited to a function of managing/displaying the results of rehabilitation exercises done by a patient, and it is thus impossible to easily understand how much the physical functions of the patient have been restored or how much the patient has become close to a healthy person. It is conventionally difficult to grasp the result of rehabilitation.

Embodiments of the present invention have been made in consideration of the above problem, and has as its object to easily grasp the result of rehabilitation.

### Means of Solution to the Problem

According to the present invention, there is provided a rehabilitation support system comprising a physical measurement unit attached to a measurement subject and configured to measure, in time series, physical information representing a static or dynamic condition of a body of the measurement subject, a physiological measurement unit configured to measure, in time series, physiological information of the inside of the body of the measurement subject, a first calculation unit configured to obtain an activity amount of the measurement subject from a change in the physical information measured by the physical measurement unit, a second calculation unit configured to obtain a physiological load imposed on the measurement subject from a change in the physiological information measured by the physiological measurement unit, a graph generation unit configured to generate a graph concerning the activity amount obtained by the first calculation unit and the physiological load obtained by the second calculation unit, and a display unit used by the measurement subject to visually recognize the graph generated by the graph generation unit.

According to embodiments of the present invention, there is also provided a rehabilitation support method comprising a first step of measuring physical information representing a static or dynamic condition of a body of a measurement subject, a second step of measuring physiological information of the inside of the body of the measurement subject, a third step of obtaining an activity amount of the measurement subject from the measured physical information, a fourth step of obtaining a physiological load imposed on the measurement subject from the measured physiological information, a fifth step of generating a graph concerning the activity amount obtained in the third step and the physiological load obtained in the fourth step, and a sixth step of visually recognizably displaying the generated graph to the measurement subject.

### Effect of Embodiments of the Invention

As described above, according to embodiments of the present invention, a graph concerning an activity amount and an exercise load which are obtained from physical information and physiological information measured in a measurement subject is displayed, thereby obtaining an excellent effect of easily grasping the result of rehabilitation.

### BRIEF DESCRIPTION OF THE DRAWINGS

FIG. 1 is a block diagram showing the arrangement of a rehabilitation support system according to the first embodiment of the present invention;

FIG. 2 is a block diagram showing a hardware arrangement in part of the rehabilitation support system according to the first embodiment of the present invention;

FIG. 3 is a flowchart for explaining an example (rehabilitation support method) of the operation of the rehabilitation support system according to the first embodiment of the present invention;

FIG. 4 is a timing chart for explaining a heart rate and the waveform of cardiac potential data;

FIG. 5 is a graph showing an example of a two-dimensional graph displayed in the rehabilitation support system according to the first embodiment of the present invention;

FIG. 6 is a graph showing statistical data concerning the relationship between a FIM and the moving standard deviation of measured acceleration;

FIG. 7 is a block diagram showing the partial arrangement of a rehabilitation support system according to the second embodiment of the present invention;

FIG. 8 is a two-dimensional graph in which a 24-hour accumulated value of % HRR is set as an exercise load and an activity time (the total sum of a standing time, a sitting time, and a walking time) in 24 hours is set as an activity amount;

FIG. 9 is a graph showing a result of measuring the posture angles of measurement subjects;

FIG. 10 shows timing charts of a change in estimated posture;

FIG. 11 is a view showing the actual measurement values of angles at the time of standing, lying face up, and lying face down;

FIG. 12 is an explanatory view for explaining a range of 300 to 1400 as a range for determining a waking position from the actual values of the angles at the time of standing, lying face up, and lying face down;

FIG. 13 is a graph showing statistical data concerning the relationship between an activity amount and a quotient obtained by dividing an exercise load by the activity amount;

FIG. 14 is a graph showing statistical data concerning the relationship between a SIAS and a quotient obtained by dividing an exercise load by an activity amount;

FIG. 15 is a timing chart showing, in time series, additional processing values each obtained by dividing an exercise load by an activity amount;

FIG. 16 is an explanatory view showing a state in which walking is detected;

FIG. 17 is an explanatory view showing a state in which two thresholds used when counting the number of steps are set in correspondence with left and right legs to ensure the accuracy of detection of walking;

FIG. 18 is a block diagram showing the arrangement of a rehabilitation support system according to the third embodiment of the present invention;

FIG. 19 is a two-dimensional graph in which the ordinate represents total exercise intensity (exercise load) in a day and the abscissa represents a total activity time (activity amount) in a day;

FIG. 20 is a graph for explaining an activity amount threshold Aₜₕ and an exercise load threshold Lₜₕ which are set for an activity amount A and an exercise load L, respectively;

FIG. 21 is a flowchart for explaining an example of the operation of the rehabilitation support system according to the third embodiment of the present invention;

FIG. 22 is an explanatory view showing an example of displaying a two-dimensional graph (a) of an exercise load and an activity amount by adding, to it, a lapse of time of the exercise load (b) and a lapse of time of the activity amount (c);

FIG. 23 is an explanatory view showing an example of displaying advice in addition to the results of the exercise load and the activity amount;

FIG. 24 is a graph showing the relationship between oxygen uptake reserve and an activity amount from walking to running of a measurement subject which is calculated by expression (4);

FIG. 25 is a graph showing the relationship between oxygen uptake reserve and the positive square root of the activity amount from walking to running of a measurement subject which is calculated by expression (4);

FIG. 26 is a graph showing a result of performing fast Fourier transform of the temporal change of the sum of accelerations in three directions measured by a physical measurement unit 101;

FIG. 27 is a graph showing the relationship between oxygen uptake reserve and the frequency of a peak obtained by performing fast Fourier transform of the temporal change of the sum of the accelerations in the three directions measured by the physical measurement unit 101;

FIG. 28 is a block diagram showing the arrangement of a rehabilitation support system according to the fourth embodiment of the present invention;

FIG. 29 is a block diagram showing the arrangement of a rehabilitation support system according to the fifth embodiment of the present invention;

FIG. 30 is a graph showing the relationship among oxygen uptake reserve, % HRR, and an activity amount obtained by the positive square root of a value calculated by expression (4) with respect to an acceleration measurement value during a period from walking to running of a measurement subject calculated by expression (4); and

FIG. 31 is a graph showing the relationship between the positive square root of an activity amount and % HRR with respect to a healthy person, in which the abscissa represents the positive square root of the activity amount and the ordinate represents % HRR.

### DETAILED DESCRIPTION OF ILLUSTRATIVE EMBODIMENTS

A rehabilitation support system according to each embodiment of the present invention will be described below.

### First Embodiment

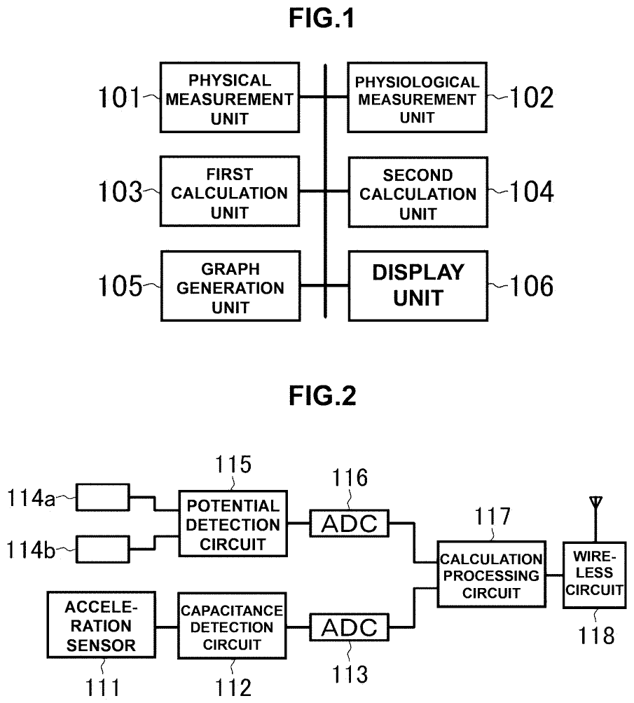

A rehabilitation support system according to the first embodiment of the present invention will first be described with reference to FIG. 1. This rehabilitation support system includes a physical measurement unit 101, a physiological measurement unit 102, a first calculation unit 103, a second calculation unit 104, a graph generation unit 105, and a display unit 106.

The physical measurement unit 101 is attached to a measurement subject (patient), and measures, in time series, physical information representing the static/dynamic condition of the body of the measurement subject. For example, the physical information includes, for example, at least one of acceleration, an angular velocity, and a position coordinate point. An acceleration measurement unit that measures acceleration in time series will be exemplified as the physical measurement unit 101 below. The physiological measurement unit 102 measures physiological information of the inside of the body of the measurement subject. The physiological information includes, for example, at least one of a cardiac potential, a heart rate, a pulse rate, a blood pressure, a myoelectric potential, and a respiratory activity. An electrocardiogram measurement unit that measures the cardiac potential of the measurement subject will be exemplified as the physiological measurement unit 102 below.

The first calculation unit 103 obtains an activity amount concerning the body movement of the measurement subject from a change in physical information measured by the physical measurement unit 101. For example, the first calculation unit 103 obtains an activity amount by one or a combination of the sum of squares, the square root of the sum of squares, the accumulated value for an arbitrary period, the time difference, the absolute value of the time difference, and the standard deviation or variance for an arbitrary period of the measured physical information.

The second calculation unit 104 obtains a physiological load imposed on the measurement subject from a change in physiological information measured by the physiological measurement unit 102. The second calculation unit 104 may obtain, for example, an exercise load as the physiological load. For example, the second calculation unit 104 may obtain an exercise load by one or a combination of the normalized value based on an arbitrary reference, the accumulated value for an arbitrary period, the average value, the median, and the differential value of the measured physiological information. The arbitrary period is set to, for example, 24 hours that include a lapse of the whole day.

The graph generation unit 105 generates a graph concerning the activity amount obtained by the first calculation unit 103 and the physiological load obtained by the second calculation unit 104. For example, the graph generation unit 105 generates a two-dimensional graph of the first parameter and the second parameter, wherein the first parameter indicates a change in activity amount obtained by the first calculation unit 103 and the second parameter indicates a change in physiological load (for example, exercise load) obtained by the second calculation unit 104. The display unit 106 visually recognizably displays, to the measurement subject, the graph of graph data generated by the graph generation unit 105.

For example, a wearable device shown in FIG. 2 may be used to transmit measurement data to a remotely arranged server via a gateway, and the server may obtain an activity amount and an exercise load, generate two-dimensional graph data including a change in obtained activity amount as the first parameter and a change in obtained exercise load as the second parameter, and display the graph of the graph data to the measurement subject.

In this arrangement, the functions of the first calculation unit 103, the second calculation unit 104, and the graph generation unit 105 are implemented in the server. The server is a computer apparatus including a CPU (Central Processing Unit), a main storage device, an external storage device, and a network connecting device, and implements the above-described functions when the CPU operates in accordance with a program loaded into the main storage device.

The device shown in FIG. 2 includes an acceleration sensor 111, a capacitance detection circuit 112, an analog/digital circuit (ADC) 113, two electrodes 114a and 114b, a potential detection circuit 115, an analog/digital circuit (ADC) 116, a calculation processing circuit 117, and a wireless circuit 118.

In the acceleration sensor 11, a movable member provided inside is displaced in accordance with a change in acceleration, thereby producing a capacitance change. This capacitance change is converted into an electrical signal by the capacitance detection circuit 112, and converted into digital data by the ADC 113, thereby obtaining acceleration data. The physical measurement unit 101 includes the acceleration sensor 11, the capacitance detection circuit 112, and the ADC 113.

The two electrodes 114a and 114b are, for example, embedded in clothing to be able to contact skin. A potential difference generated between the two electrodes 114a and 114b is detected by the potential detection circuit 115, and converted into digital data by the analog/digital circuit (ADC) 116, thereby obtaining cardiac potential data. The electrodes 114a and 114b, the potential detection circuit 115, and the ADC 116 serve as the physiological measurement unit 102.

The calculation processing circuit 117 acquires acceleration data and cardiac potential data at every set time (for example, per second). The acceleration data and the cardiac potential data acquired by the calculation processing circuit 117 are transmitted by the wireless circuit 118 to a server via a gateway (not shown).

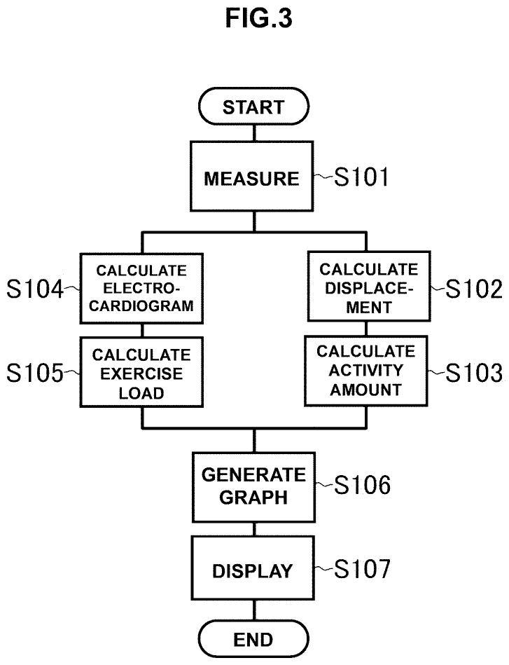

An example (rehabilitation support method) of the operation of the rehabilitation support system according to the first embodiment will be described next with reference to a flowchart shown in FIG. 3.

In step S101, the physical measurement unit 101 measures a capacitance change as physical information, for example, as a change in acceleration, and the physiological measurement unit 102 measures a potential difference as physiological information (first and second steps). Next, in step S102, the physical measurement unit 101 calculates a displacement from the measured capacitance change, and sets it as acceleration data. In step S103, the first calculation unit 103 obtains an activity amount associated with the body movement of the measurement subject from the acceleration data (third step).

In step S104, the physiological measurement unit 102 calculates an electrocardiogram from the measured potential difference, and sets it as the cardiac potential of the measurement subject. In step S105, the second calculation unit 104 obtains the exercise load of the measurement subject from the cardiac potential (fourth step).

In step S106, the graph generation unit 105 generates data of a graph concerning the activity amount obtained in the third step and the physiological load obtained in the fourth step (fifth step). For example, the graph generation unit 105 sets a change in obtained activity amount as the first parameter and a change in obtained exercise load as the second parameter, and generates data of a two-dimensional graph in which the abscissa represents the first parameter and the ordinate represents the second parameter. In step S107, the display unit 106 displays the generated graph (two-dimensional graph) (sixth step).

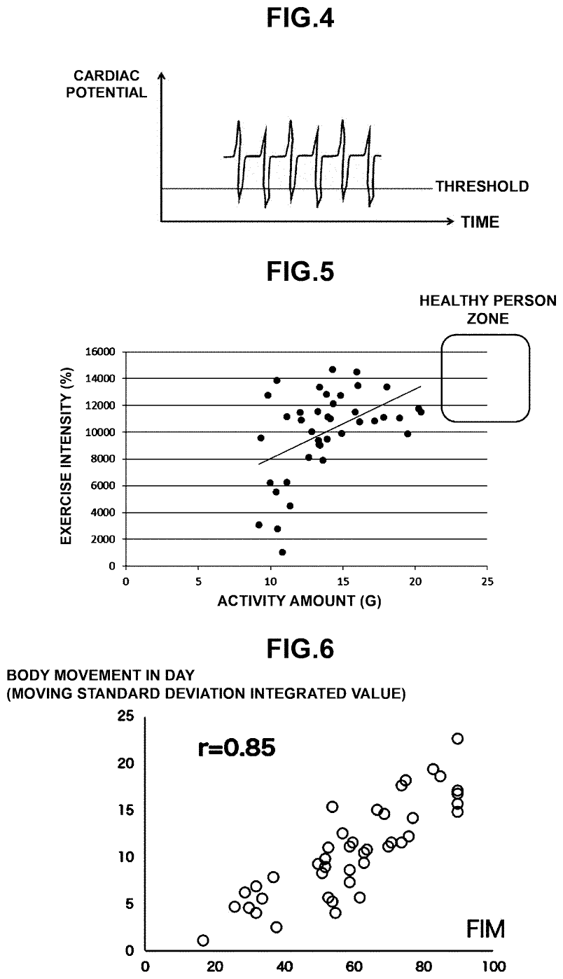

Calculation of the exercise load will now be described. The exercise load can be obtained from a heart rate. The heart rate can be calculated as, for example, the number of peaks per minute by performing, using a predetermined threshold, threshold processing of the waveform of cardiac potential data to detect a peak and measuring a time interval from the peak to the next peak (see FIG. 4). A value obtained by dividing this heart rate by the maximum heart rate of the measurement subject is set as the exercise load. Note that “measured heart rate/maximum heart rate of measurement subject×100” is generally called exercise intensity (% MHR; Maximum Heart Rate).

The exercise load may be obtained using the difference (heart rate reserved; HRR) between the resting heart rate and the maximum heart rate by “(measured heart rate−resting heart rate of measurement subject)/(maximum heart rate of measurement subject−resting heart rate)”. This calculation result is called exercise intensity (% HRR; % Heart Rate Reserve).

The activity amount obtained from the acceleration will be described next. The acceleration is detected using a three-axis acceleration sensor that detects accelerations in three directions along X-, Y-, and Z-axes. Since the acceleration of each axis changes depending on the tilt of the physical measurement unit 101 attached to the measurement subject, for example, a norm (composition of vectors) is used as a displacement. The norm is given by “|a|=(aₓ²+a_{y}²+a_{z}²)^{1/2}” when aₓ, a_{y}, and a_{z} represent the accelerations in the directions along the x-, y-, and z-axes, respectively. The use of the square root increases the calculation amount, and thus the sum (aₓ²+a_{y}²+a_{z}²) of squares may be used. To remove an unintended extremely large vibration in measured acceleration, a low-pass filter may be applied to the norm or the sum of squares.

The activity amount is calculated by integrating the norm (moving total), as given by expression (1) below.

Σᵢ₌₁^{k}|aᵢ| (1)

The measurement results of the exercise amount and activity amount obtained as described above are used to generate a two-dimensional graph (see FIG. 5) in which the ordinate represents the exercise intensity (exercise load) and the abscissa represents the activity amount. In FIG. 5, the measurement results are indicated by circles. In this graph, a healthy person zone obtained from a data group of healthy persons measured in advance is set and displayed. Thus, it is possible to visually grasp how much the measurement results indicated by open circles are close to the healthy person zone. In addition, it is possible to consider the restorative effect of rehabilitation in linkage with (in association with) the physical functions.

The activity amount may be calculated by integrating the squared value of the difference in temporal change of the norm, as given by expression (2) below.

Σᵢ₌₁^{k}(|aᵢ₊₁|−|aᵢ|)² (2)

The activity amount may be calculated by integrating the absolute value of the difference in temporal change of the norm (the sum of the absolute values of the differences), as given by expression (3) below. In this case, the calculation amount can be reduced, as compared with the sum of squares of the difference.

Σᵢ₌₁^{k}∥aᵢ₊₁−|a_{∥} (3)

The activity amount may be calculated using the moving standard deviation as the standard deviation of the temporal change of the norm, as given by expression (4) below.

$$\sqrt{\frac{1}{k} {\sum }_{i = 1}^{k} {( \vert \overline{a} \vert - \vert {a}_{i} \vert )}^{2}} \qquad ( 4 )$$

FIG. 6 shows statistical data concerning the relationship between a FIM (Functional Independence Measure) and the moving standard deviation. The FIM is an evaluation index of ADL (Activities of Daily Living). It is found that the moving standard deviation has a correlation with the FIM. With respect to the above-described activity amount, the same value (1G) as the gravitational acceleration is output at rest. However, since the moving standard deviation is an amount representing a deviation from an average, a very small value is output at rest. The use of the moving standard deviation can give an index reflecting an exercise.

### Second Embodiment

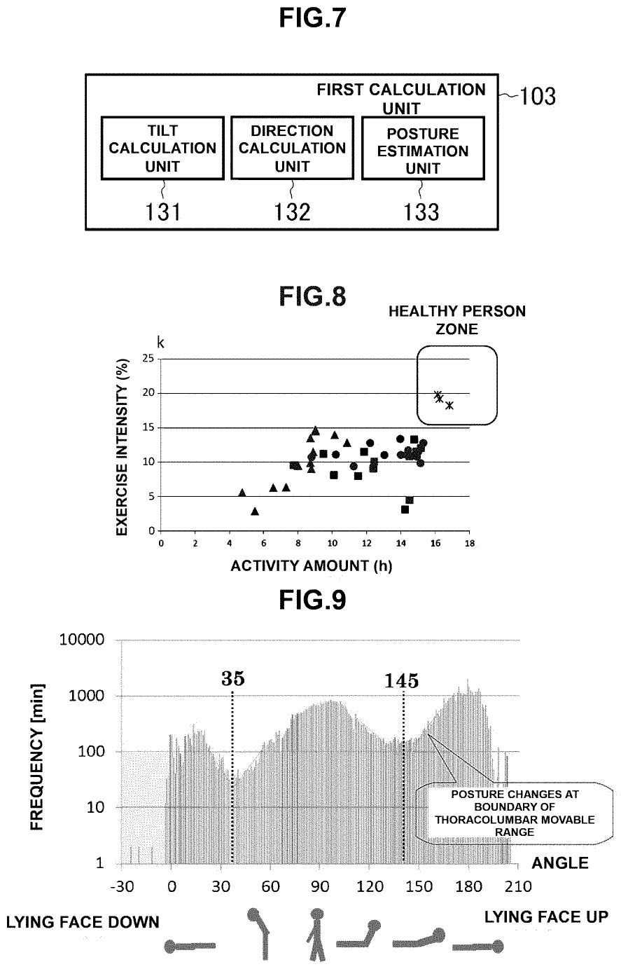

A rehabilitation support system according to the second embodiment of the present invention will be described next. In the second embodiment, a first calculation unit 103 estimates the posture of a measurement subject from acceleration measured by a physical measurement unit 101, and sets it as an activity amount. In the second embodiment, as shown in FIG. 7, the first calculation unit 103 includes a tilt calculation unit 131, a direction calculation unit 132, and a posture estimation unit 133.

The tilt calculation unit 131 obtains an angle θ of the tilt of the measurement subject from the acceleration measured by the physical measurement unit 101, as given by equation (5) below.

$$θ = - \frac{π}{180} {cos}^{- 1} ( \frac{{a}_{z , i}}{\vert {a}_{i} \vert} ) \qquad ( 5 )$$

The direction calculation unit 132 obtains a direction θ of the measurement subject from the acceleration measured by the physical measurement unit 101, as given by equation (6) below.

$$φ = - \frac{π}{180} {cos}^{- 1} ( \frac{{a}_{z , i}}{\vert {a}_{i} \vert} ) \qquad ( 6 )$$

Note that θ (−90≤θ<270) represents the tilt of the z-axis of the physical measurement unit 101 with respect to the vertical direction, ϕ (−90≤ϕ<270) represents the tilt of the x-axis of the physical measurement unit 101 with respect to the vertical direction, and the unit for θ and ϕ is ° [degree].

The posture estimation unit 133 estimates the posture by comparing the values of the angle θ and direction ϕ of the tilts obtained as described above with thresholds. The tilt of the physical measurement unit 101 reflects the tilt of the upper body of the measurement subject to which the physical measurement unit 101 is attached, and thus the posture of the measurement subject can be estimated from the tilt of the physical measurement unit 101.

FIG. 8 is a two-dimensional graph in which a 24-hour accumulated value of % HRR is set as exercise intensity (exercise load) and an activity time (the total sum of a standing time, a sitting time, and a walking time) in 24 hours is set as an activity amount. In FIG. 8, the measurement results of patients are indicated by circles, rectangles, and triangles. The circles indicate patients each having a high FIM, the rectangles indicate patients each having a medium FIM, and the triangles indicate patients each having a low FIM. In this graph, a healthy person zone (*) obtained from a data group of healthy persons measured in advance is set and displayed. In FIG. 8, as the FIM is higher, the patient is closer to the healthy person zone, and it is thus understood that the measurement result and the FIM as the existing medical evaluation index of rehabilitation have a correlation. This makes it possible to visually grasp how much the measurement results indicated by the circles are close to the healthy person zone. In addition, it is possible to consider the restorative effect of rehabilitation in linkage with (in association with) the physical functions.

FIG. 9 shows a result of measuring the posture angles of patients (measurement subjects). This is a result of performing measurement for 48 hours for each of 28 measurement subjects (including males and females). For example, it is possible to set a highly reliable threshold based on a lifestyle in a hospital by statistically searching for an angle at which the posture is switched.

The total time of a sitting time, a standing time, and a walking time among the estimated postures, that is, the total time excluding a sleeping (lying) time is set as an activity amount. In consideration of the posture, the accuracy of the activity amount can be improved.

A standard deviation s of the acceleration may be obtained from the acceleration measured by the physical measurement unit 101, as given by equation (7) below, and the direction obtained by the direction calculation unit 132 may be compensated by the standard deviation s.

$$S = \sqrt{\frac{1}{n}} {\sum }_{i = 1}^{k} {( \vert \overline{a} \vert - \vert {a}_{i} \vert )}^{2} \qquad ( 7 )$$

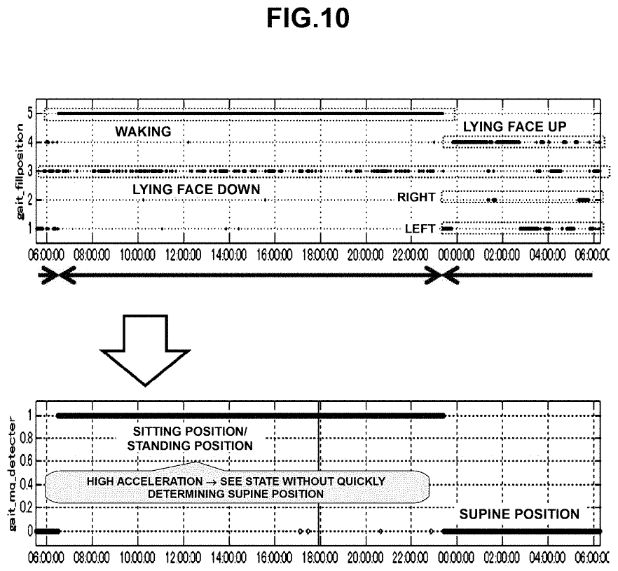

For example, as shown in FIG. 10, if the acceleration measured by the physical measurement unit 101 is high, it is interpreted as a sitting position or a standing position. If the acceleration measured by the physical measurement unit 101 is low, it is interpreted as a supine position, and the direction of the body obtained by the direction calculation unit 132 is held for a predetermined time. It is possible to perform stable posture estimation which is strong against disturbance by holding the direction while paying attention to the magnitude of the acceleration measured by the physical measurement unit 101.

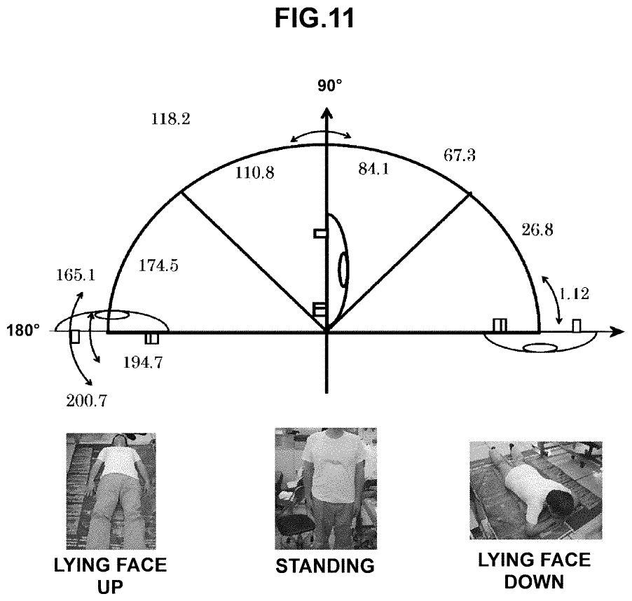

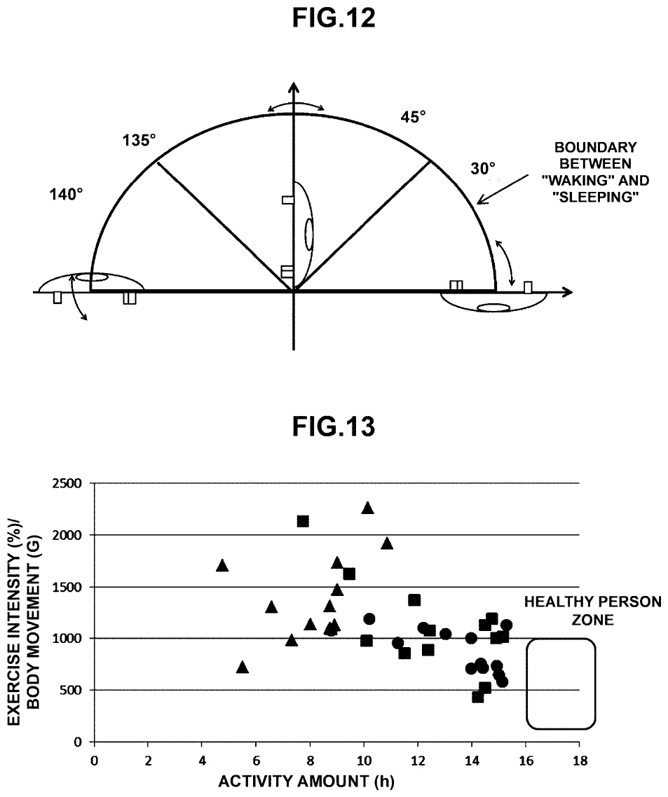

The actual measurement values of angles at the time of standing, lying face up, and lying face down will be described below with reference to FIGS. 11 and 12. As shown in FIG. 11, a result falling within the range of 165° to 200° is obtained at the time of lying face up, a result falling within the range of 1° to 27° is obtained at the time of lying face down, and a result falling within the range of 67° to 118° is obtained at the time of standing. Based on an experiment result, a range for determining a waking position is set to the range of 30° to 140° (FIG. 12). When the threshold of the posture is decided based on the statistical distribution of the patients, as described above, an actual life in a hospital can be simulated, thereby improving the estimation accuracy of the posture.

The second calculation unit 104 may obtain an additional processing value by dividing the obtained exercise load by the activity amount obtained by the first calculation unit 103, and the graph generation unit 105 may generate a two-dimensional graph of the first parameter and the second parameter that indicates a change in additional processing value obtained by the second calculation unit 104. For example, the graph shown in FIG. 5 may be changed to a graph shown in FIG. 13 in which a quotient (additional processing value) obtained by dividing the exercise load by the activity amount is used as the exercise intensity (exercise load) on the ordinate. In FIG. 13, circles indicate patients each having a high FIM, rectangles indicate patients each having a medium FIM, and triangles indicate patients each having a low FIM.

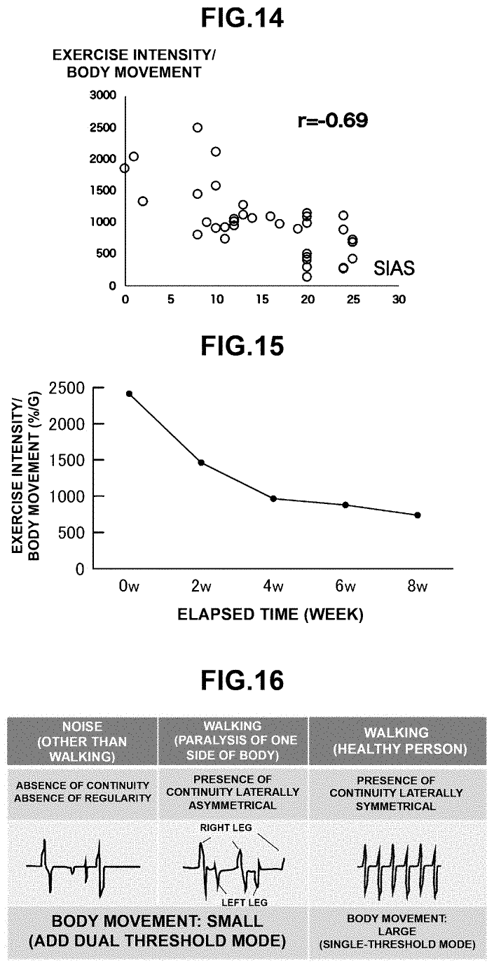

FIG. 14 shows statistical data concerning the relationship between a SIAS (Stroke Impairment Assessment Set) as an evaluation index of function impairment used to treat apoplexy and a 24-hour accumulation of % HRR divided by the activity amount obtained from the moving standard deviation. As shown in FIG. 14, it is apparent that the index on the ordinate has an inverse correlation with the SIAS. The exercise intensity (exercise load) divided by the activity amount serves as an index of efficiency when a patient moves the body, and can thus be used for the ordinate to evaluate the relationship between the activity amount as the activity time and the efficiency of a body operation.

The graph generation unit 105 generates a graph (time-series graph) that displays, in time series, the additional processing values obtained by the second calculation unit 104, as exemplified in FIG. 15, and the display unit 106 may display this time-series graph. FIG. 15 shows transition of the load (exercise intensity/body movement) of the body movement, that is, the body operation of each of a plurality of patients as the additional processing value. By displaying the time-series graph on the display unit 106, it can be confirmed that the load decreases along with a lapse of weeks as a result of rehabilitation. A decrease in load means that the patient can move the body easily, and indicates that the efficiency is improved. The additional processing values are worth to the time-series information, and are thus displayed on the display unit 106 in time series to be able to support rehabilitation appropriately. Note that the graph generation unit 105 may generate both a time-series graph showing the additional processing values in time series and the above-described two-dimensional graph and display them on the display unit 106 at the same time, or the graph generation unit 105 may generate one of the above graphs and display the generated graph on the display unit 106.

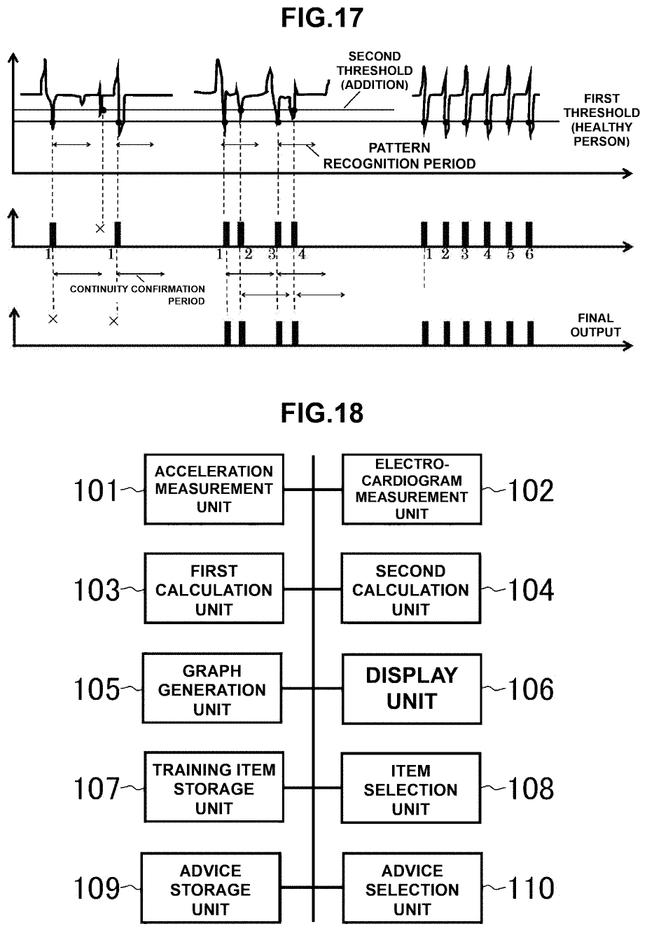

For a patient with apoplexy, paralysis of the lower half of the body may occur. In this case, a body movement is different between the right and left legs, and it is impossible to obtain sufficient accuracy of detection of walking by a pedometer generally used (see FIG. 16). In this case, as shown in FIG. 17, two thresholds used when counting the number of steps are set in correspondence with the left and right legs, and it is thus possible to ensure the accuracy of detection of walking even for a patient with paralysis of one side of the body.

### Third Embodiment

A rehabilitation support system according to the third embodiment of the present invention will be described next with reference to FIG. 18. In the third embodiment, a training item storage unit 107 and an item selection unit 108 are further provided in the rehabilitation support system according to the first embodiment.

The training item storage unit 107 stores a plurality of items concerning rehabilitation in association with activity amounts and exercise loads. The item selection unit 108 selects one of the items stored in the training item storage unit 107 based on an activity amount obtained by a first calculation unit 103 and an exercise load obtained by a second calculation unit 104. The item selected by the item selection unit 108 is displayed on a display unit 106 together with a graph generated by a graph generation unit 105.

The rehabilitation support system according to the third embodiment further includes an advice storage unit 109 and an advice selection unit 110.

The advice storage unit 109 stores a plurality of pieces of advice concerning rehabilitation in association with activity amounts and exercise loads. The advice selection unit 110 selects one of the pieces of advice stored in the advice storage unit 109 based on the activity amount obtained by the first calculation unit 103 and the exercise load obtained by the second calculation unit 104. The piece of advice selected by the advice selection unit 110 is displayed on the display unit 106 together with the graph generated by the graph generation unit 105.

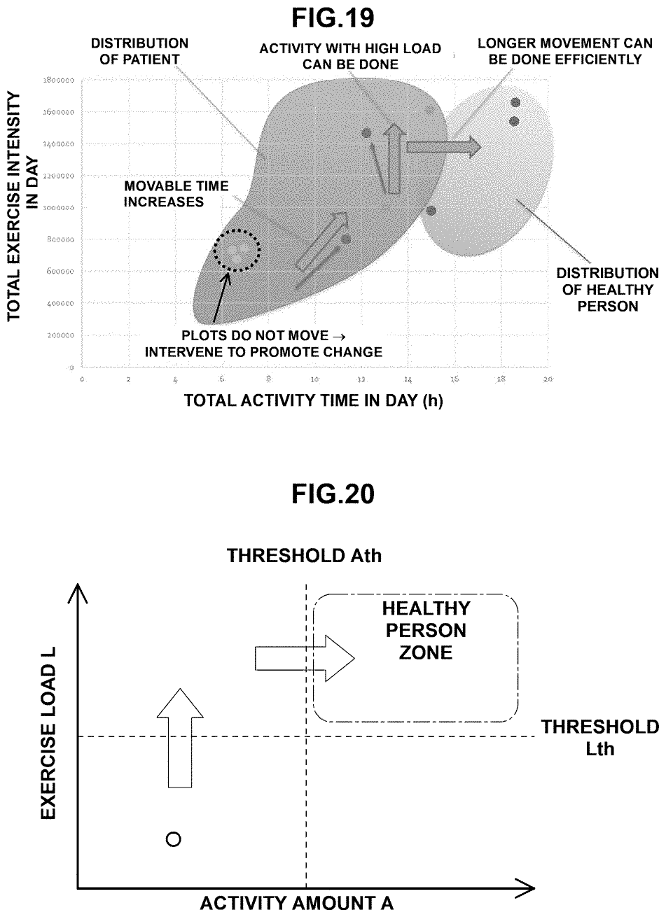

For example, FIG. 19 shows a two-dimensional graph in which the ordinate represents the total exercise intensity (exercise load) in a day and the abscissa represents the total activity time (activity amount) in a day. As the total exercise intensity in a day, “(measured heart rate−resting heart rate of measurement subject)/(maximum heart rate of measurement subject−resting heart rate)×100” is used, and as the total activity time in a day, a total time in a day during which the posture other than a sleeping or lying position is obtained is used. As shown in FIG. 19, it is apparent that as the exercise load and the activity amount are larger, the movable time increases, an activity with a higher load can be done, and longer movement can be done efficiently.

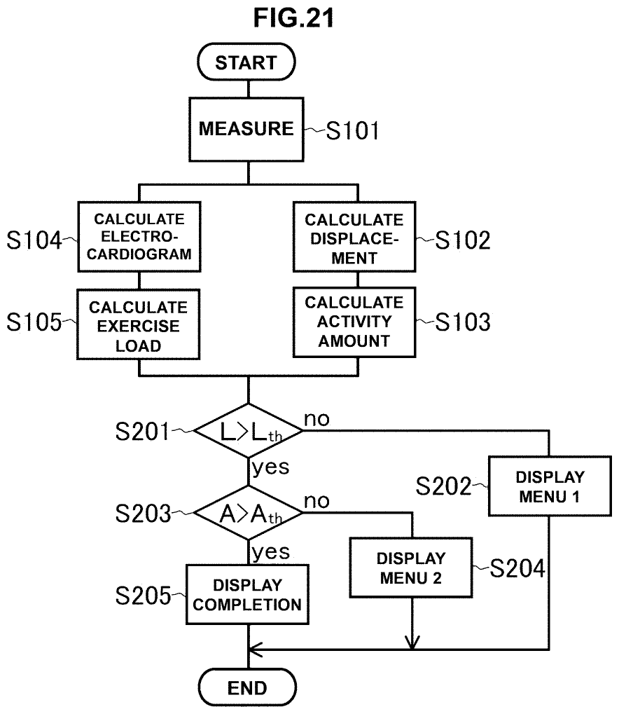

As shown in FIG. 20, with respect to an activity amount A and an exercise load L, an activity amount threshold Aₜₕ and an exercise load threshold Lₜₕ are set based on a healthy person zone. Under this condition, based on a flowchart shown in FIG. 21, the obtained exercise load and activity amount undergo threshold determination, thereby making it possible to present a rehabilitation menu as training items suitable for the condition of a patient.

In step S101, a physical measurement unit 101 measures a capacitance change as a change in acceleration, and a physiological measurement unit 102 measures a potential difference. In step S102, the physical measurement unit 101 calculates a displacement from the measured capacitance change, and sets it as acceleration data. In step S103, the first calculation unit 103 obtains an activity amount associated with the body movement of a measurement subject from the acceleration data.

In step S104, the physiological measurement unit 102 calculates an electrocardiogram from the measured potential difference, and sets it as the cardiac potential of the measurement subject. In step S105, the second calculation unit 104 obtains the exercise load of the measurement subject from the cardiac potential.

In step S201, the item selection unit 108 determines whether the obtained exercise load L is larger than the threshold Lₜₕ. If the exercise load L is equal to or smaller than the threshold Lₜₕ (NO in step S201), the item selection unit 108 selects, in step S202, menu 1 from the training item storage unit 107, and displays it on the display unit 106. On the other hand, if the exercise load L is larger than the threshold Lₜₕ (YES in step S201), the process advances to step S203, and the item selection unit 108 determines whether the obtained activity amount A is larger than the threshold Aₜₕ. If the activity amount A is equal to or smaller than the threshold Aₜₕ (NO in step S203), the item selection unit 108 selects, in step S202, menu 2 from the training item storage unit 107, and displays it on the display unit 106. On the other hand, if the activity amount A is larger than the threshold Aₜₕ (YES in step S203), the process advances to step S205, and the item selection unit 108 displays a notification of completion of rehabilitation on the display unit 106.

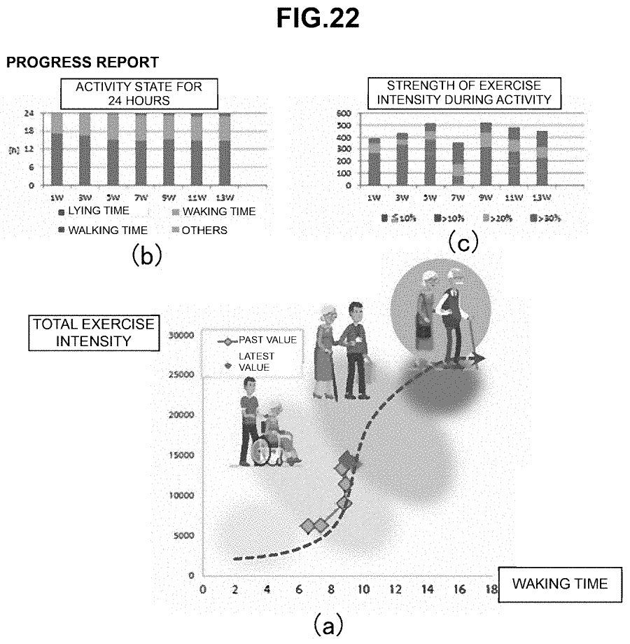

As shown in FIG. 22, in addition to a two-dimensional graph (a) of the exercise load and the activity amount, the lapse of time of the exercise load (b) and the lapse of time of the activity amount (c) may be displayed. This allows a patient to grasp an operation in daily life in association with the exercise load and the activity amount, and feed it back to the currently performed rehabilitation.

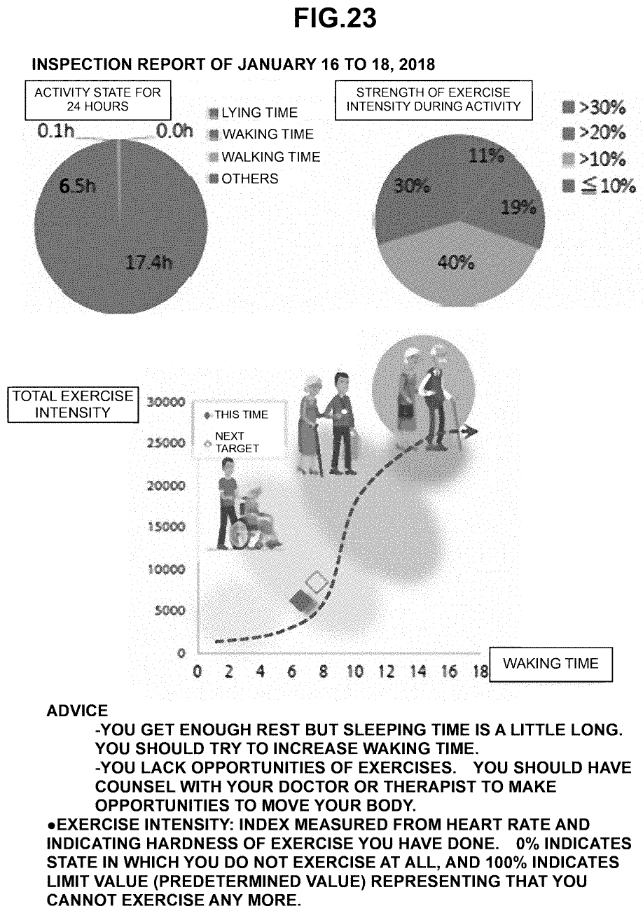

An example of presentation of advice will be described next. For example, as shown in FIG. 23, advice is displayed in addition to the results of the exercise load and activity amount. Advice given by a doctor or the like is stored in the advice storage unit 109. The advice selection unit 110 selects, using an algorithm such as machine learning for the results of the obtained exercise load and activity amount, a piece of advice as a typical document stored in the advice storage unit 109, and displays it on the display unit 106. This can present, to the patient, improvements of the currently performed rehabilitation.

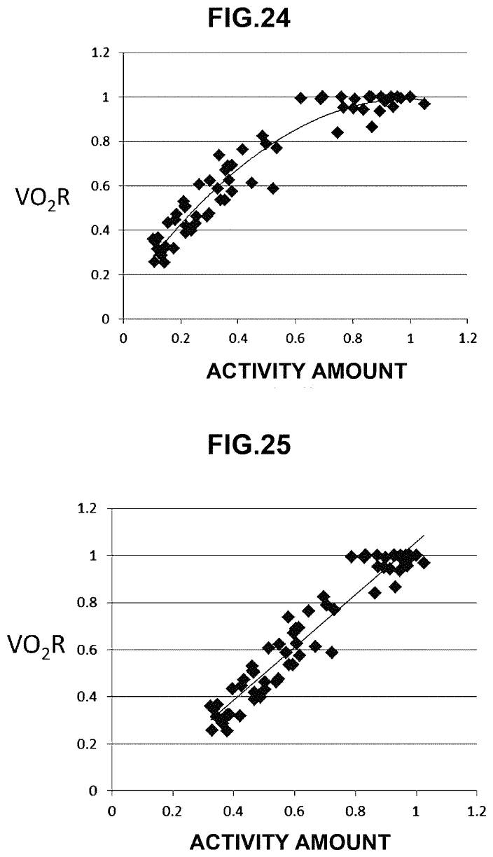

As the activity amount, the positive square root of an activity amount calculated by expression (1), (2), (3), or (4) can be used. FIG. 24 shows the relationship between oxygen uptake reserve and an activity amount from walking to running of the measurement subject which is calculated by expression (4). FIG. 25 shows the relationship between oxygen uptake reserve and the positive square root of an activity amount from walking to running of the measurement subject which is calculated by expression (4). Plots are quadratically, nonlinearly distributed in FIG. 24 while plots are linearly distributed in FIG. 25. This trend is the same even if the maximum oxygen uptake is used instead of the oxygen uptake reserve. Furthermore, the same trend is obtained even if expression (1), (2), or (3) is used instead of expression (4).

The linear relationship has advantages that it is readily processed intuitionally to predict an oxygen uptake and the calculation amount is small, and can highly reliably be applied to analysis assuming linearity, for example, multiple regression analysis.

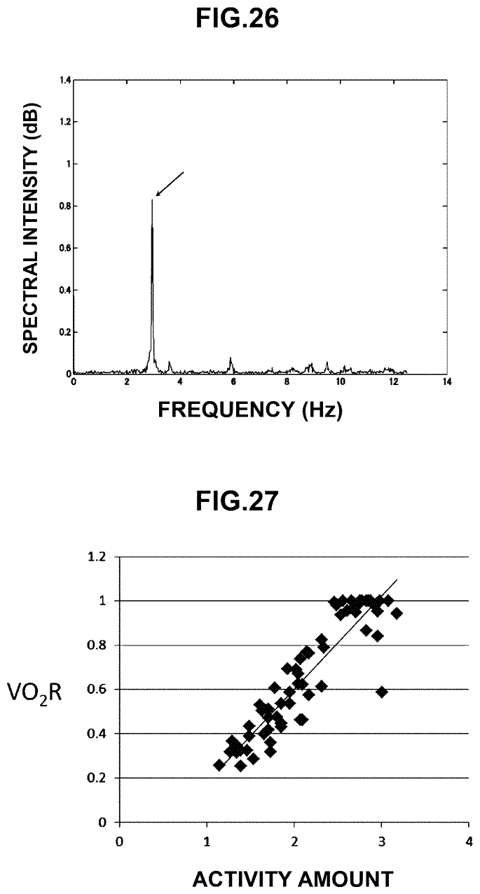

The peak frequency of the temporal change of the sum of accelerations in three directions measured by the physical measurement unit 101 can be used as the activity amount. FIG. 26 shows a result of performing fast Fourier transform (FFT) of the sum of the accelerations in the three directions measured by the physical measurement unit 101 at temporally consecutive 1,024 points, that is, the temporal change for 40.96 sec at a data rate of 25 Hz. There is a peak at 3 Hz, from which it is found that the patient runs at a pitch of 3 steps/sec, that is, a pitch of 180 steps/min. FIG. 27 shows the relationship between oxygen uptake reserve and the frequency of the peak, and it is found that the correlation is obtained. This indicates the relationship between the walk pitch and the oxygen uptake, and it is possible to simultaneously grasp the detailed condition of walking or running and an oxygen uptake in the condition.

### Fourth Embodiment

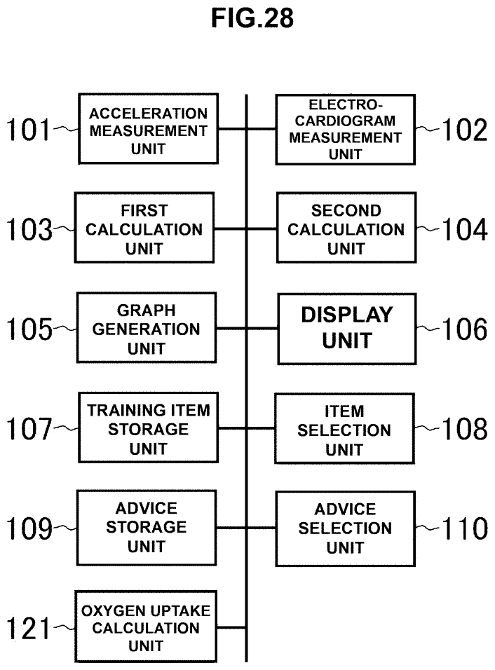

A rehabilitation support system according to the fourth embodiment of the present invention will be described next with reference to FIG. 28. In the fourth embodiment, an oxygen uptake calculation unit 121 is further provided in the rehabilitation support system according to the third embodiment. The oxygen uptake calculation unit 121 creates a regression equation from the distribution of an activity amount and an oxygen uptake, and calculates an oxygen uptake using the created regression equation. The regression equation may be created using a distribution obtained in advance, or may be created every time from an activity amount and an oxygen uptake stored in the oxygen uptake calculation unit 121.

A case in which the distribution obtained in advance is used will be described by exemplifying the distributions shown in FIGS. 24, 25, and 27. For example, the regression equation of the distribution shown in FIG. 24 is given by “Y=−0.9X²+1.79X+0.11” when Y represents oxygen uptake reserve and X represents an activity amount. The regression equation of the distribution shown in FIG. 25 is given by “Y=1.12X−0.06” when Y represents oxygen uptake reserve and X represents an activity amount. The regression equation of the distribution shown in FIG. 27 is given by “Y=0.42X−0.24” when Y represents oxygen uptake reserve and X represents an activity amount. The oxygen uptake calculation unit 121 can implement these regression equations.

The oxygen uptake is actually measured from expiration but expiration measurement places a heavy burden on a measurement subject. Therefore, it is possible to grasp an oxygen uptake with a small burden by simply estimating an oxygen uptake from an activity amount using the above-described regression equation.

### Fifth Embodiment

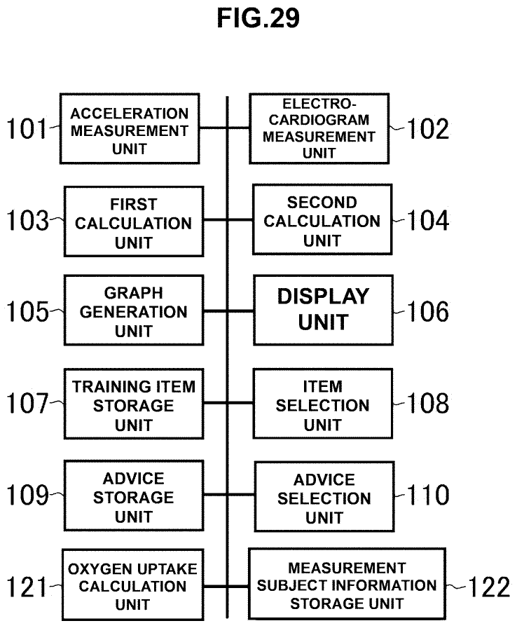

A rehabilitation support system according to the fifth embodiment of the present invention will be described next with reference to FIG. 29. In the fifth embodiment, a measurement subject information storage unit 122 is further provided in the rehabilitation support system according to the fourth embodiment. The measurement subject information storage unit 122 stores at least one of pieces of history information of a measurement subject including the date of birth, age, sex, height, weight, case history, medication history, hospitalization history, person in charge of treatment, FIM (Functional Independence Measure), sick room, bed used of the measurement subject. By providing the measurement subject information storage unit 122 that stores the history information of a measurement subject, a factor in a change in exercise load or activity amount of the measurement subject can be taken into consideration.

An oxygen uptake calculation unit 121 can obtain a maximum oxygen uptake or oxygen uptake reserve from at least one of an activity amount obtained by a first calculation unit 103, a physiological load obtained by a second calculation unit 104, and the history information of the measurement subject stored in the measurement subject information storage unit 122.

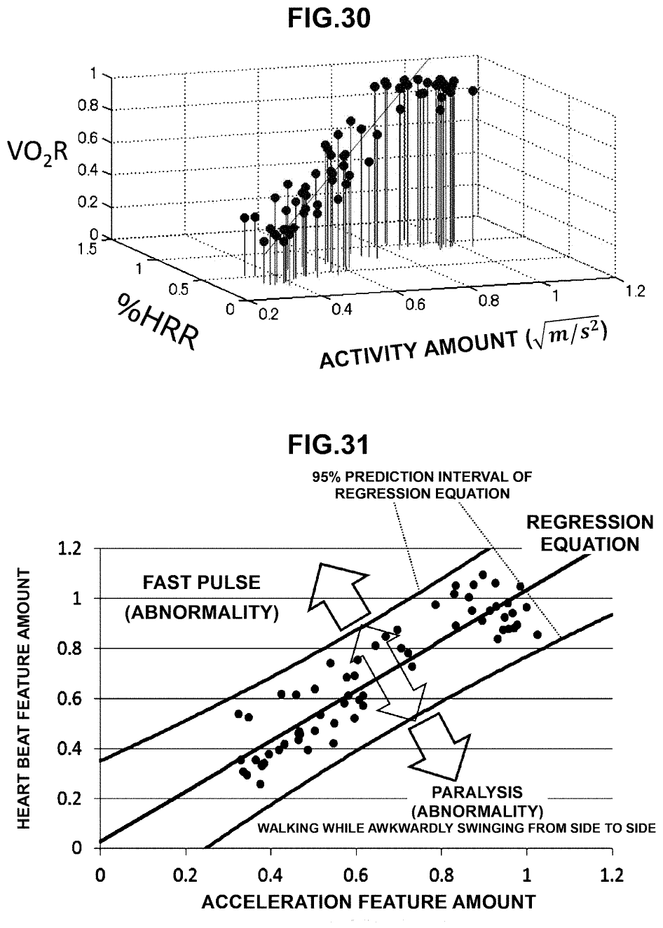

FIG. 30 shows the relationship among oxygen uptake reserve, % HRR, and an activity amount obtained from the positive square root of a value calculated by expression (4) with respect to an acceleration measurement value during a period from walking to running of a measurement subject calculated by expression (4). It is found that the activity amount and % HRR each have a correlation with the oxygen uptake reserve. This correlation may be used to create the above-described regression equation. An equation for multiple regression analysis is generally given by Y=β₀+Σᵢ₌₁βᵢxᵢ (i=1, 2, 3, . . . ) However, when Y represents the oxygen uptake reserve, x₁ represents an exercise load, and x₂ represents the positive square root of an activity amount, the regression equation is given by “Y=0.39x₁+0.71x₂−0.07 . . . (8)” using multiple regression analysis.

Equation (8) does not include the history information of the measurement subject. However, if multiple regression analysis is performed using the history information as a subsequent term, a regression equation including the history information can be obtained. If the number of exercise loads xᵢ is large, only x₁ having a strong relationship with Y may be selected using a stepwise variable selection method (see non-patent literature 1) to create a regression equation. The stepwise variable selection method can be performed mechanically, and can thus readily be implemented in the system.

Table 1 shows a coefficient R² of determination of the regression equation of the oxygen uptake reserve obtained using the positive square root of the activity amount, the coefficient R² of determination of the regression equation of the oxygen uptake reserve obtained using % HRR, the coefficient R² of determination of the regression equation of the oxygen uptake reserve obtained by multiple regression analysis using both the positive square root of the activity amount and % HRR. It is found that when both the positive square root of the activity amount and % HRR are used, most satisfactory estimation accuracy is obtained. By using a multivariate regression equation, it is possible to provide a correct estimation value of an oxygen uptake.

TABLE 1

| | R² |
|---|---|
| % HRR | 0.88 |
| activity amount | 0.92 |
| multiple regression model | 0.94 |

For the multivariate regression equation, logistic regression, support vector regression, and a neural network can be used instead of multiple regression analysis. Since these can perform nonlinear regression that cannot be performed by multiple regression analysis, more optimized regression can be performed, thereby providing a highly reliable estimation value of an oxygen uptake.

Furthermore, in the multivariate regression equation, each term can be multiplied by a coefficient, and the value of the coefficient can be switched in accordance with a condition. For example, coefficients a and b (0≤a, b≤1) are given, like “Y=β₀+aβ₁x₁+bβ₂x₂=0.39ax₁+0.71bx₂−0.07”, and the values of these coefficients are switched in accordance with a condition. An example of switching will be described with reference to FIG. 31. FIG. 31 shows the relationship between the positive square root of the activity amount and % HRR with respect to a healthy person, in which the abscissa represents the positive square root of the activity amount and the ordinate represents % HRR. Although data points vary due to the individual differences and measurement errors with respect to a regression line, the 95% prediction interval is calculated, thereby making it possible to grasp that the variation statistically remains within the range.

However, unlike healthy persons, data may appear at a position exceeding the 95% prediction interval for a patient. For example, for a patient having a fast pulse, % HRR is high and thus the 95% prediction interval is exceeded. On the other hand, if the swing of the body in walking is large due to excess paralysis, the positive square root of the activity amount is large, and thus the 95% prediction interval is exceeded. In these cases, the value of one of the terms of the exercise load and the activity amount is an abnormal value. Therefore, if the above regression equation is used, the term of the abnormal value leads to a deterioration in reliability, and thus the use of the above regression equation is not appropriate.

On the other hand, if % HRR is high and thus the 95% prediction interval is exceeded in FIG. 31, a=0 is set in the above equation to eliminate its contribution, and thus the term of the abnormal value does not lead to a deterioration in reliability, thereby allowing appropriate estimation. Similarly, if the positive square root of the activity amount is high and thus the 95% prediction interval is exceeded, b=0 is set in the above equation to eliminate its contribution, and thus the term of the abnormal value does not lead to a deterioration in reliability, thereby allowing appropriate estimation. If data falls within the 95% prediction interval, a=b=1 is set. In this way, each term is multiplied by a coefficient, and the value of the coefficient is switched in accordance with a condition, thereby making it possible to provide a highly reliable estimation value of an oxygen uptake even for a person with disease.

As described above, according to embodiments of the present invention, a graph concerning the activity amount and the exercise load obtained from the physical information and the physiological information measured in the measurement subject is displayed, and it is thus easy to grasp the effect of rehabilitation.

In the rehabilitation support system, an angular velocity sensor (gyro sensor) may be used as the physical measurement unit. The angular velocity sensor outputs, as a measurement value, an angle as an alternative to θ or ϕ described above, and can thus advantageously acquire the activity amount more easily. A GPS may be used as the physical measurement unit. The GPS acquires position information, and can thus calculate a moving amount from the history of the position information, thereby providing a moving amount that is effective in terms of motion monitoring.

An electromyograph may be used as the physiological measurement unit. While it is possible to grasp the metabolism of the entire body including the central nervous system and the peripheral nervous system by the electrocardiograph, it is possible to measure a local myoelectric potential by the electromyograph, and provide restricted load information that facilitates analysis. Alternatively, a respirometer may be used as the physiological measurement unit. If the exercise load increases, a respiration rate also generally increases. Thus, the respirometer is expected to play the role similar to the electrocardiograph, and it is expected to substitute a respiration rate for a heart rate. In addition, since the sensor of the respirometer need not be arranged on the skin surface of the body, it is easy to attach/detach the respirometer.

Alternatively, a sphygmomanometer is used as the physiological measurement unit. If a person exercises, oxygen consumption increases, and blood pressure also increases similar to the heart rate, thereby making it possible to substitute the blood pressure for the heart rate. If, for example, the blood pressure is always measured due to disease or the like, the use of another sensor is complicated and it is thus possible to ensure convenience by using the sphygmomanometer currently used. A pulse monitor may be used as the physiological measurement unit. If a pulse rate is used, it can be measured at an arm, leg, neck, or the like where it is difficult to measure a cardiac potential, thereby implementing easier measurement.

Note that the present invention is not limited to the above-described embodiments, and it is obvious that many modifications and combinations can be made by a person with normal knowledge in the field within the technical scope of the present invention.

### EXPLANATION OF THE REFERENCE NUMERALS AND SIGNS

101 . . . physical measurement unit, 102 . . . physiological measurement unit, 103 . . . first calculation unit, 104 . . . second calculation unit, 105 . . . graph generation unit, 106 . . . display unit.

1.-17. (canceled)

18. A rehabilitation support system comprising:
a physical measuring device attached to a measurement subject and configured to measure, in time series, physical information representing a static or dynamic condition of a body of the measurement subject;
a physiological measuring device configured to measure, in time series, physiological information in the body of the measurement subject;
a processor configured to:
obtain an activity amount of the measurement subject from a change in the physical information measured by the physical measuring device;
obtain a physiological load imposed on the measurement subject from a change in the physiological information measured by the physiological measuring device; and
generate a graph concerning the activity amount and the physiological load; and
a display device configured to display the graph.

19. The rehabilitation support system according to claim 18, wherein:
the physical information measured by the physical measuring device includes acceleration, an angular velocity, or a position coordinate point; and
the physiological information measured by the physiological measuring device includes a cardiac potential, a heart rate, a pulse rate, a myoelectric potential, or respiration.

20. The rehabilitation support system according to claim 18, wherein:
the physical measuring device comprises an acceleration measuring device configured to measure acceleration in time series;
the physiological measuring device comprises an electrocardiogram measurement unit configured to measure a cardiac potential of the measurement subject; and
the physiological load is an exercise load.

21. The rehabilitation support system according to claim 20, wherein the exercise load is obtained according to a measured heart rate/a maximum heart rate of the measurement subject, (the measured heart rate−a resting heart rate of the measurement subject)/(the maximum heart rate of the measurement subject−the resting heart rate), an accumulated value for an arbitrary period, or an average value or median for the arbitrary period.

22. The rehabilitation support system according to claim 20, wherein:
the acceleration measuring device measures accelerations in three directions along X-, Y, and Z-axes orthogonal to each other; and
the activity amount is obtained from an integrated value of a sum of the accelerations in the three directions, an integrated value of a squared value of a difference in a temporal change of the sum of the accelerations in the three directions, an integrated value of an absolute value of the difference in the temporal change of the sum of the accelerations in the three directions, or a moving standard deviation of the temporal change of the sum of the accelerations in the three directions.

23. The rehabilitation support system according to claim 22, wherein the activity amount is obtained by calculating a positive square root of the integrated value of the sum of the accelerations in the three directions, the integrated value of the squared value of the difference in the temporal change of the sum of the accelerations in the three directions, the integrated value of the absolute value of the difference in the temporal change of the sum of the accelerations in the three directions, or the moving standard deviation of the temporal change of the sum of the accelerations in the three directions.

24. The rehabilitation support system according to claim 20, wherein:
the acceleration measuring device measures accelerations in three directions along X-, Y, and Z-axes orthogonal to each other; and
a tilt angle of an upper body of the measurement subject is obtained from the accelerations and the activity amount is a posture of the measurement subject decided by the tilt angle.

25. The rehabilitation support system according to claim 20, wherein:
the acceleration measuring device measures accelerations in three directions along X-, Y, and Z-axes orthogonal to each other; and
the activity amount is obtained by obtaining a peak frequency of a temporal change of a sum of the accelerations in the three directions.

26. The rehabilitation support system according to claim 20, wherein:
an additional processing value is obtained by dividing the exercise load by the activity amount; and
a two-dimensional graph of a first parameter and a second parameter is generated using a change in the activity amount as a first parameter and a change in the additional processing value as a second parameter.

27. The rehabilitation support system according to claim 20, wherein:
an additional processing value is obtained by dividing the exercise load by the activity amount; and
a graph that represents the additional processing values in time series is generated.

28. The rehabilitation support system according to claim 18, wherein a two-dimensional graph of a first parameter and a second parameter is generated, using a change in the activity amount as the first parameter and a change in the physiological load as the second parameter.

29. The rehabilitation support system according to claim 18, further comprising:
a training item storage device configured to store a plurality of items concerning rehabilitation in association with activity amounts and physiological loads;
wherein the processor is further configured to select a first item of the plurality of items stored in the training item storage device based on the activity amount obtained and the physiological load; and
the display device is further configured to display the first item together with the graph.

30. The rehabilitation support system according to claim 18, further comprising:
an advice storage device configured to store a plurality of pieces of advice concerning rehabilitation in association with activity amounts and exercise loads;
wherein the processor further configured to o select a first piece of advice of the plurality of pieces of advice stored in the advice storage device based on the activity amount and the physiological load; and
the display device is further configured to display the first piece of advice together with the graph.

31. The rehabilitation support system according to claim 18, further comprising an oxygen uptake calculator configured to obtain a maximum oxygen uptake or an oxygen uptake reserve from the activity amount o or the physiological load.

32. The rehabilitation support system according to claim 18, further comprising a measurement subject information storage device configured to store history information of the measurement subject including a date of birth, an age, a sex, a height, a weight, a case history, a medication history, a hospitalization history, a person in charge of treatment, a FIM, a sick room, or a bed used of the measurement subject.

33. The rehabilitation support system according to claim 32 further comprising an oxygen uptake calculator configured to obtain a maximum oxygen uptake or an oxygen uptake reserve from the activity amount, the physiological load, or the history information of the measurement subject.

34. A rehabilitation support method comprising:
measuring physical information representing a static or dynamic condition of a body of a measurement subject;
measuring physiological information of the inside of the body of the measurement subject;
obtaining, by a processor of a device, an activity amount of the measurement subject from the physical information;
obtaining, by the processor, a physiological load imposed on the measurement subject from the physiological information;
generating, by the processor, a graph concerning the activity amount and the physiological load; and
displaying, on a display of the device, the graph.
